(19) United States
(12) Patent Application Publication
(10) Pub. No.: US 2009/0234173 A1
(43) Pub. Date: Sep. 17, 2009

(54) ORGANIC WASTE DECOMPOSITION SYSTEM AND METHOD WITH BLOWER

(76) Inventor: Myeong Yurl Lee, Siheung-si (KR)

Correspondence Address: KNOBBE MARTENS OLSON & BEAR LLP, 2040 MAIN STREET, FOURTEENTH FLOOR, IRVINE, CA, 92614, US

(21) Appl. No.: 12/405,921

(22) Filed: Mar. 17, 2009

Related U.S. Application Data
(60) Provisional application No. 61/037,134, filed on Mar. 17, 2008.

(51) Int. Cl. A62D 3/40; A61L 2/04

(52) U.S. Cl. 588/321; 422/307

(57) ABSTRACT

A system and a method of decomposing organic waste are provided. The system decomposes organic waste in a decomposition chamber without use of enzymes, additives, or microorganisms. In one embodiment, the system decomposes organic waste within 24 hours and deodorizes the odor of decomposing organic waste during decomposition process. The system provides sufficient heat and operating conditions to evaporate moisture from the organic waste without burning the organic waste. The byproduct of the organic waste after decomposition process by the system is substantially homogeneous material that is reduced in volume compared to the organic waste. In one embodiment, the system reuses or recycles water and heat used in the system for different processes in the system. The system includes a blower that provides flow of the moisture inside the system.

### CROSS-REFERENCE TO RELATED APPLICATION

This application claims the benefit to U.S. Provisional Patent Application No. 61,037,134 filed Mar. 17, 2008. Also, this application relates to U.S. Non-provisional patent application Ser. No. ______, filed concurrently herewith, entitled “ORGANIC WASTE DECOMPOSITION SYSTEM AND METHOD WITH WATER RECYCLING” (Attorney Docket No. CTP.001A) and further relates to U.S. Non-provisional patent application Ser. No. ______, filed concurrently herewith, entitled “ORGANIC WASTE DECOMPOSITION SYSTEM AND METHOD WITH HEAT RECYCLING” (Attorney Docket No. CTP.001A2).

### BACKGROUND

1. Field

The present disclosure is in the field of waste management system, particularly in the field of organic waste decomposition system.

2. Related Technology

Recent federal statistics indicate that nearly 65 million tons of food or organic wastes are generated annually in the United States, but only 3% of those are composted or given as animal feed. The remainder is disposed in landfills or incinerators. Composting of organic waste is considered an environmentally friendly treatment by naturally decomposing the biodegradable organic waste. The organic waste can be broken down by microorganisms and the broken down waste or by-product can be used as soil amendments, such as fertilizers. However, decomposition process takes a long time. Thus, there is a need for more effective processes and systems to decompose organic waste.

### SUMMARY

One aspect of the invention provides an organic waste decomposition system. The system may comprise: a decomposition chamber configured to decompose organic waste therein such that the decomposition chamber heats the organic waste to release moisture therefrom and continue to heat the moisture and organic waste to decompose, whereby steam is generated in the decomposition chamber; a condenser in fluid communication with the decomposition chamber via a conduit and configured to precipitate water from the steam passing therethrough; a blower in fluid communication with the decomposition chamber and the condenser, the blower being configured to flow steam from the decomposition chamber to the condenser; a water circuit in fluid communication between the condenser and the decomposition chamber, the water circuit configured to supply precipitated water to the decomposition chamber.

The foregoing system may further comprise: a filter between the decomposition chamber and the condenser, the filter being configured to screen debris from the decomposition chamber; and a flusher configured to flush debris deposited on the filter. The flusher may be in fluid communication with the water circuit and configured to use the at least part of the precipitated water for flushing. The water circuit may comprise a water tank configured to at least temporarily store precipitated water therein. The water circuit may comprise a water pump configured to flow precipitated water from the water tank toward the decomposition chamber. The water circuit may comprise a water filter configured to filter at least part of the precipitated water that may be discharged from the system. The water filter may comprise an activated carbon filter. The water circuit may be configured to supply precipitated water to the decomposition chamber when the organic waste may be in shortage of moisture for decomposing.

Another aspect of the invention provides a method of operating an organic waste decomposition system using the foregoing systems. The method may comprise: decomposing organic waste in a decomposition chamber such that the decomposition chamber heats the organic waste to release moisture therefrom and continue to heat the moisture and organic waste to decompose, whereby steam may be generated in the decomposition chamber; condensing the steam passing through a condenser in fluid communication with the decomposition chamber via a conduit to precipitate water; flowing steam from the decomposition chamber to the condenser with a blower in fluid communication with the decomposition chamber and the condenser; supplying precipitated water to the decomposition chamber by a water circuit in fluid communication between the condenser and the decomposition chamber.

The foregoing method may further comprise: filtering by screening debris from the decomposition chamber with a filter between the decomposition chamber and the condenser; and flushing debris deposited on the filter with a flusher. The flusher may be in fluid communication with the water circuit and configured to use the at least part of the precipitated water for flushing. The water circuit may comprise a water tank configured to at least temporarily store precipitated water therein. The water circuit may comprise a water pump configured to flow precipitated water from the water tank toward the decomposition chamber. The water circuit may comprise a water filter configured to filter at least part of the precipitated water that may be discharged from the system. The water filter may comprise an activated carbon filter. The water circuit may be configured to supply precipitated water to the decomposition chamber when the organic waste may be in shortage of moisture for decomposing.

Another aspect of the invention provides an organic waste decomposition system. The system comprises: a decomposition chamber configured to decompose organic waste therein such that the decomposition chamber heats the organic waste to release moisture therefrom and continue to heat the moisture and organic waste to decompose, whereby steam is generated in the decomposition chamber; a condenser in fluid communication with the decomposition chamber via a conduit and configured to precipitate water from the steam passing therethrough; a blower in fluid communication with the decomposition chamber and the condenser, the blower being configured to flow steam from the decomposition chamber to the condenser; a deodorizer in fluid communication with the condenser to receive vapor that may have passed the condenser and configured to remove odor from the vapor; and a vapor circuit in fluid communication between the deodorizer and decomposition chamber and configured to send to the decomposition chamber at least part of the vapor that may have passed the deodorizer.

In the foregoing system, the deodorizer may comprise a metallic catalyst and a heater configured to heat the vapor to a temperature from about 200° C. to about 500° C. The vapor circuit may comprise a heat insulated conduit configured to flow therethrough the vapor that may have been heated through the deodorizer. The vapor circuit may comprise an air inlet configured to receive and send ambient air to the decomposition chamber. The system may further comprise: a heating member configured to provide heat; and a liquid medium configured to be heated by the heating member and heat the decomposition chamber with the heat retained therein. The vapor circuit may be configured to send at least part of the vapor that may have passed the deodorizer to the liquid medium.

Another aspect of the invention provides a method of operating an organic waste decomposition system. The method comprises: decomposing organic waste in a decomposition chamber such that the decomposition chamber heats the organic waste to release moisture therefrom and continue to heat the moisture and organic waste to decompose, whereby steam may be generated in the decomposition chamber; condensing the steam passing through a condenser in fluid communication with the decomposition chamber via a conduit to precipitate water; flowing steam from the decomposition chamber to the condenser with a blower in fluid communication with the decomposition chamber and the condenser; removing odor from vapor that may have passed the condenser with a deodorizer in fluid communication with the condenser; and sending at least part of the vapor that may have passed the deodorizer to the decomposition chamber with a vapor circuit in fluid communication between the deodorizer and decomposition chamber.

In the foregoing method, the deodorizer may comprise a metallic catalyst and a heater configured to heat the vapor to a temperature from about 200° C. to about 500° C. The vapor circuit may comprise a heat insulated conduit configured to flow therethrough the vapor that may have been heated through the deodorizer. The vapor circuit may comprise an air inlet configured to receive and send ambient air to the decomposition chamber. The method may further comprise: heating a liquid medium; and heating the decomposition chamber with the heat retained in the liquid medium. The vapor circuit may be configured to send at least part of the vapor that may have passed the deodorizer to the liquid medium.

Still another aspect of the invention provides an organic waste decomposition system. The system comprises: a decomposition chamber configured to decompose organic waste therein such that the decomposition chamber heats the organic waste to release moisture therefrom and continue to heat the moisture and organic waste to decompose, whereby steam is generated in the decomposition chamber; a condenser in fluid communication with the decomposition chamber via a conduit and configured to precipitate water from the steam passing therethrough; and a blower in fluid communication with the decomposition chamber and the condenser, the blower being configured to flow steam from the decomposition chamber to the condenser, wherein the blower is set to flow the steam at a flow rate such that a relative humidity within the decomposition chamber is maintained with less than 10% variation for at least several hours.

In the foregoing system, the decomposition chamber may have an interior volume, wherein the blower may be set to create the flow rate, which may be from about 2 to about 4 times the interior volume for a minute. The system may further comprise an air inlet configured to receive and send ambient air to the decomposition chamber. The blower and the air inlet may be set to create flow rate of steam and send ambient air to the decomposition chamber, respectively, to maintain the relative humidity inside the decomposition chamber at about 90% for at least several hours. The blower may be configured to flow and discharge at least part of the steam from the condenser. The blower may be configured to flow at least part of the steam from the condenser to the decomposition chamber.

A still further aspect of the invention provides a method of operating an organic waste decomposition system. The method comprises: decomposing organic waste in a decomposition chamber such that the decomposition chamber heats the organic waste to release moisture therefrom and continue to heat the moisture and organic waste to decompose, whereby steam is generated in the decomposition chamber; condensing the steam passing through a condenser in fluid communication with the decomposition chamber via a conduit to precipitate water; and flowing the steam from the decomposition chamber to the condenser with a blower in fluid communication with the decomposition chamber and the condenser, wherein the blower is set to flow the steam at a flow rate such that a relative humidity within the decomposition chamber is maintained with less than 10% variation for at least several hours.

In the foregoing method, the decomposition chamber may have an interior volume, wherein the blower may be set to create the flow rate, which may be from about 2 to about 4 times the interior volume for a minute. The method may further comprise receiving and sending ambient air to the decomposition chamber with an air inlet. The blower and the air inlet may be set to create flow rate of steam and send ambient air to the decomposition chamber, respectively, to maintain the relative humidity inside the decomposition chamber at about 90% for at least several hours. The blower may be configured to flow and discharge at least part of the steam from the condenser. The blower may be configured to flow at least part of the steam from the condenser to the decomposition chamber.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only some embodiments in accordance with the disclosure and are, therefore, not to be considered limiting of its scope, the disclosure will be described with additional specificity and detail through use of the accompanying drawings.

FIG. 1 is a block diagram of an organic waste decomposing system according to one embodiment.

FIG. 2 is a more detailed illustration of the organic waste decomposing system of FIG. 1.

FIG. 3 is a block diagram of a control system of an organic waste decomposing system according to one embodiment.

FIG. 4 illustrates flow of moisture during an organic waste decomposing process according to one embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

In some embodiments, a system and a method of decomposing organic waste are provided. In some embodiments, the system is configured to decompose organic waste in a decomposition chamber without use of enzymes, additives, or microorganisms and produce by-products have potential use as bio-mass and/or bio-fuel. In some embodiments, the system may be configured to decompose organic waste within 24 hours and deodorizes odor of the decomposing organic waste during decomposition process. The system is configured to provide sufficient heat and operating conditions to evaporate moisture from the organic waste without burning the organic waste. The system is configured to reuse or recycle water and heat used in the system for different processes in the system.

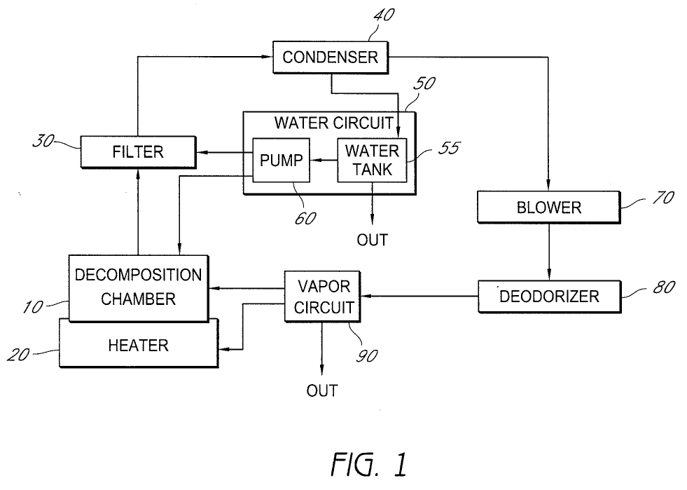

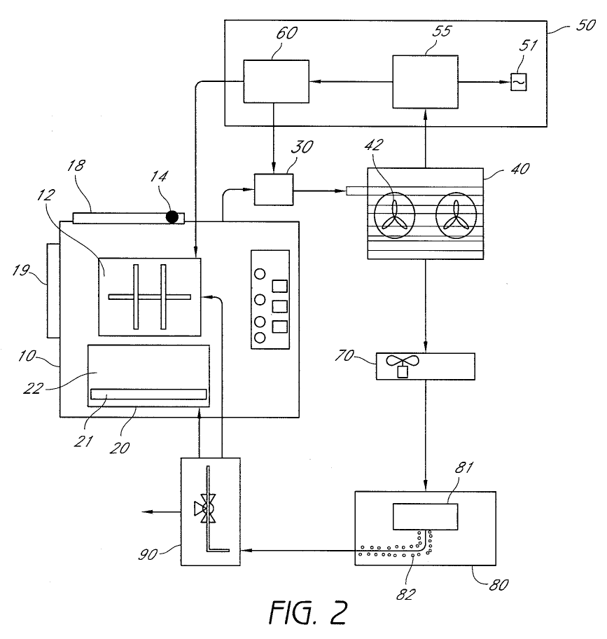

Referring to an illustrated embodiment as shown in FIGS. 1 and 2, the organic waste decomposition system (OWDS) includes a decomposition chamber 10, a heater 20, a filter 30, a condenser 40, a blower 70, a water circuit 50, a deodorizer 80, and a vapor circuit 90.

### Decomposition Chamber

The decomposition chamber 10 is where organic waste is decomposed. In one embodiment, the decomposition chamber 10 has an input door 18 for inputting or loading organic waste into the decomposition chamber 10. In one embodiment, an emergency stop switch 14 is provided such that the input door 18 can press the emergency stop switch 14 when the input door 18 is closed. However, when the input door 18 is open and the emergency stop switch 14 is unpressed, the operation of the OWDS is stopped. The decomposition chamber 10 further includes an output door 19 for outputting byproducts of the decomposition process. The capacity of the reaction chamber 10 can be determined by the amount of organic waste a user wants to decompose in a given time period. In some embodiments, the capacity or mass of the organic waste that the decomposition chamber 10 can be from about 10 kg to about 5000 kg, such as from about 30 kg to about 1500 kg. In embodiments, the decomposition chamber 10 can be substantially sealed when the input door 18 and the output door 19 are closed, except for tubing connected with other components of the OWDS which will be further described below.

In embodiments, the OWDS operates the organic waste decomposition process without burning or carbonization of the organic waste. To avoid burning or carbonization, the speed of dehydration from the decomposition chamber 10 has to be regulated. In embodiments, while the moisture content inside the decomposition chamber 10 constantly decreases during the operation, the OWDS maintains the inside of the decomposition chamber 10 humid until the organic waste is substantially decomposed and the volume has been substantially decreased.

For example, at least for several hours (about 3, about 4, about 5, about 6, about 7, about 8, about 9, about 10, about 11, about 12, about 13, about 14, about 15 hours) of operation, the relative humidity inside the decomposition chamber 10 stays above about 75%, about 76%, about 77%, about 78%, about 79%, about 80%, about 81%, about 81%, about 82%, about 83%, about 84%, about 85%, about 86%, about 87%, about 88%, about 89%, and about 90%, about 91%, about 92%, about 93%, about 94%, about 95%, about 96%, about 97%, about 98%, about 99%, and about 100%. Also, for example, at least for several hours (about 3, about 4, about 5, about 6, about 7, about 8, about 9, about 10, about 11, about 12, about 13, about 14, about 15 hours), the humidity inside the decomposition chamber 10 stays above about 75%, about 76%, about 77%, about 78%, about 79%, about 80%, about 81%, about 81%, about 82%, about 83%, about 84%, about 85%, about 86%, about 87%, about 88%, about 89%, and about 90%, about 91%, about 92%, about 93%, about 94%, about 95%, about 96%, about 97%, about 98%, about 99%, and about 100% of the highest humidity of the operation within the decomposition chamber 10.

### Crusher

When organic waste includes large pieces or chunks, making them smaller in size can help the decomposition process of the OWDS. In some embodiments, the OWDS can include a crusher or a dicer that is configured to crush or dice the organic waste into smaller parts before getting decomposed. The crusher may be located inside or outside the decomposition chamber 10.

### Impeller

In embodiments, the OWDS includes an impeller 12 rotatable within the decomposition chamber 10 for mixing organic waste being processed in the decomposition chamber 10. Rotation of the impeller 12 can be controlled or regulated to distribute the organic waste substantially evenly within the decomposition chamber 10. The organic waste on the bottom is driven by the impeller paddles towards the top of the chamber 10 and dropped down due to gravity. In one embodiment, the impeller 12 can include a rotation bar with one or more arms or paddles extended from the rotation bar. The one or more arms are configured to mix the organic waste. In some embodiments, the one or more arms can be perpendicular to the rotation bar or attached at an angle to the rotation bar. The impeller 12 can be configured and/or controlled to rotate in one direction during decomposing process to help distribute the organic waste and rotate in the other direction to push the decomposed waste out after the decomposing process.

### Heater

In the illustrated embodiment as shown in FIGS. 1 and 2, the heater 20 is configured to provide heat to the decomposition chamber 10. The heater 20 can include a heating member 21 and a liquid medium 22 to indirectly heat the decomposition chamber 10. The liquid medium 22 can include oil, water, etc. The liquid medium 22 generally has a high heat retention property to retain heat therein. In one embodiment, at least some portion of the heater 20 contacts the decomposition chamber 10 to heat the decomposition chamber 10. In another embodiment, the heating member 21 can heat the decomposition chamber 10 directly.

In one embodiment, the heating member 21 can heat the liquid medium 22 to a temperature from about 110° C. to about 150° C., such as about 120° C. to about 140° C. The heater 20 is controlled to heat the decomposition chamber 10 such that the temperature inside the decomposition chamber 10 can be at about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, or about 105° C. The temperature inside the decomposition chamber 10 is maintained within a range formed by two of the numbers listed in the immediately previous sentence.

The foregoing temperature ranges within the decomposition chamber 10 can decompose the organic waste without burning them when sufficient moisture is provided within the decomposition chamber 10. As the heater 20 heats the decomposition chamber 10 over time, moisture within organic waste in the decomposition chamber 10 starts to evaporate and released into the decomposition chamber 10. In some embodiments, heating of the heater 20 can be controlled by a controller of the OWDS throughout the process. The heater 20 can controllably heat the decomposition chamber 10 in order to desiccate the organic waste without burning them. In one embodiment, the heater 20 can be turned off and on in order to maintain a certain temperature range within the decomposition chamber 10. Although not limited, the heater 20 uses electricity for generating heat.

### Filter

In the illustrated embodiment as shown in FIGS. 1 and 2, the filter 30 is disposed near or at a steam outlet of the decomposition chamber 10, where steam is discharged from the decomposition chamber 10. In embodiments, the steam through the filter 30 is directed to the condenser 40 through at least one conduit or pipe. The filter 30 screens debris or dirt that is mixed within moisture coming from the decomposition chamber 10. If the debris from organic waste is not filtered by the filter 30, they can cause clogging in the conduit or pipe, and also they can reach the condenser 40 and/or a water tank of the water circuit 50 and cause clogs therein. In one embodiment, the filter 30 can include at least one porous screen.

In some embodiments, the filter 30 may include more than one screen with varying sizes of pores. Size of the pores of the filter screens can vary from a few millimeters to a few microns. In one embodiment, the filter 30 can be flushed with water to wash away debris trapped on the screen(s) of the filter 30. The water to flush the filter 30 can be provided from an external source or recycled from the water tank of the water circuit 50. In one embodiment, the filter 30 can be replaced periodically to remove used clogged filter 30.

### Condenser

In the illustrated embodiment as shown in FIGS. 1 and 2, the condenser 40 is in fluid communication with the decomposition 10 and configured to receive the steam from the decomposition chamber 10. The condenser 40 is to precipitate or condense at least some of the steam coming from the decomposition chamber 10 into water. The steam minus the condensed water (or vapor) through the condenser 40 can be provided to the deodorizer 80 via blowing of the blower 70. In some embodiments, the condenser 40 condenses moisture contained in the steam at a condensation rate of about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, or about 90%. In embodiments, at least one of temperature at the condenser 40 and flowing speed of the steam through the condenser 40 is set or controlled to achieve a range of condensation rate defined by any two numbers listed in the immediately previous sentence.

In one embodiment, the condenser 40 can include at least one pipe 41 for allowing flow of steam therethrough. In some embodiments, the at least one pipe 41 can have fins running spirally, straight, or across on an exterior of the at least one pipe for better heat conduction with the cooler external environment. The condenser 40 can further include one or more cooling fans 42 for blowing air to cool the at least one pipe 41 of the condenser 40. The condensed water from the condenser 40 can be sent to a water reservoir or a water tank of the water circuit 50.

### Water Circuit

In the illustrated embodiment as shown in FIGS. 1 and 2, the water circuit 50 is configured to collect condensed moisture (water) from the condenser 40 and distribute the water to various components. The water circuit 50 can include a water tank 55 and a water pump 60. The water tank 55 is to at least temporarily store water condensed from the condenser 40. The water pump 60 is to pump or flow precipitated water from the water tank 55 towards various components of the OWDS.

In one embodiment, the water circuit 50 is in fluid communication with the condenser 40 and the decomposition chamber 10. The water circuit 50 supplies precipitated water from the condenser 40 to the decomposition chamber 10 when extra moisture is needed within the decomposition chamber 10 for the organic waste to decompose without burning. The water pump 60 is to pump the water to the decomposition chamber 10. This process can be controlled by a controller of the OWDS. In another embodiment, the water circuit 50 is in further connection with the filter 30 and supplies the water the filter 30 for flushing the filter screen(s). The water in the water tank 55 can be pumped by the water pump 60. Again, flushing can be controlled by a controller of the OWDS.

In one embodiment, at least some of the water stored in water tank 55 can be discarded to outside of the OWDS. The water discarded to outside is pre-processed through a water filter 51 to minimize pollution. The water filter 51 can include activated carbon filters. Although not illustrated, water from an external source can be supplied to the OWDS for flushing the filter 30 and/or for supplementing moisture to the decomposition chamber 10 when needed. The water from the external source can be pumped into the OWDS by the water pump 60.

### Blower

In the illustrated embodiment as shown in FIGS. 1 and 2, the blower 70 is configured to control flow of air or steam through pipes and components of the OWDS. In the illustrated embodiment, the blower 70 is generally in fluid communication with the decomposition chamber 10, the condenser 40, and the deodorizer 80, and drives the flow of the steam and vapor from the decomposition chamber 10 to the condenser 40 to the deodorizer 80. In one embodiment, the blower 70 can flow at least some of the vapor to discharge to outside of the OWDS.

In one embodiment, the blower 70 is set or controlled to flow the steam from the decomposition chamber 10 at a flow rate that can maintain certain desired level of humidity inside the chamber 10 through the decomposition process. In one embodiment, the flow rate is controlled or set such that the relative humidity within the decomposition chamber 10 is maintained with less than about 20% variation, such as about 19, about 18, about 17, about 16, about 15, about 14, about 13, about 12, about 11, about 10, about 9, about 8, about 7, about 6, and about 5% variation, for at least several hours, for example, at least about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27 hours. In one embodiment, the blower 70 is controlled to substantially continuously flow the steam out of the decomposition chamber 10. In one embodiment, the flow rate is set at a constant rate for at least several hours.

In some embodiments, flow rate of air or steam controlled by the blower 70 can be related to a size or capacity of the decomposition chamber 10. In one embodiment, the flow rate of air controlled by the blower 70 can be generally proportional to the size of the reaction chamber 10. The blower 70 can be set to create the flow rate, which is, for example, from about 2 to about 4 times the interior volume of the decomposition chamber 10 for 1 minute. In one embodiment, the flow rate of air controlled or set by the blower 70 at about 800 liter/min, about 2800 liter/min, about 4000 liter/min, about 8000 liter/min, and about 12000 liter/min for decomposition chamber capacities of about 220 L, about 850 L, about 1250 L, about 2500 L, and about 3900 L, respectively.

### Deodorizer

In the illustrated embodiment as shown in FIGS. 1 and 2, the deodorizer 80 is in fluid communication with the condenser 40 and the blower 70 to receive vapor (some steam that has passed the condenser 40). The deodorizer 80 processes vapor to remove odor before discharging to outside the system. In embodiments, the deodorizer 80 includes a catalyst 82 that is effective in removing odor from the vapor. For example, the catalyst 82 includes Pt, Ni, Ru, Rh, Pd, Ag, Fe, Co, and Ir. In one embodiment, the deodorizer 80 further includes a deodorizer heater 81 to heat the vapor to a temperature at which the catalyst 82 is active or activated. For example, the deodorizer heater 81 is configured to heat the vapor to a temperature from about 200° C. to about 500° C.

### Vapor Circuit

In the illustrated embodiment as shown in FIGS. 1 and 2, the vapor circuit 90 is in fluid communication between the deodorizer 80 and the heater 20. The connection from the deodorizer 80 to the vapor circuit 90 can be insulated so as to substantially minimize loss of heat of the vapor. In one embodiment, the conduit from the deodorizer 80 to the vapor circuit 90 can include a ceramic carrier 83.

The vapor circuit 90 recycles the heat produced in the deodorizer 80 by returning heated vapor from the deodorizer chamber 10 or by using the heated vapor to heat the liquid medium 22. In one embodiment, the vapor circuit 90 includes a heat insulated conduit to flow therethrough the vapor that has been heated through the deodorizer 80. The vapor circuit 90 can further include an air flow controller or valve 91 that regulates receiving of ambient air and/or release vapor. In one embodiment, the combination of the vapor and the ambient air can be provided to the decomposition chamber 10. In one embodiment, the vapor circuit 90 is configured to send at least part of vapor that has passed the deodorizer 80 to the liquid medium of the heater 20.

### Control System

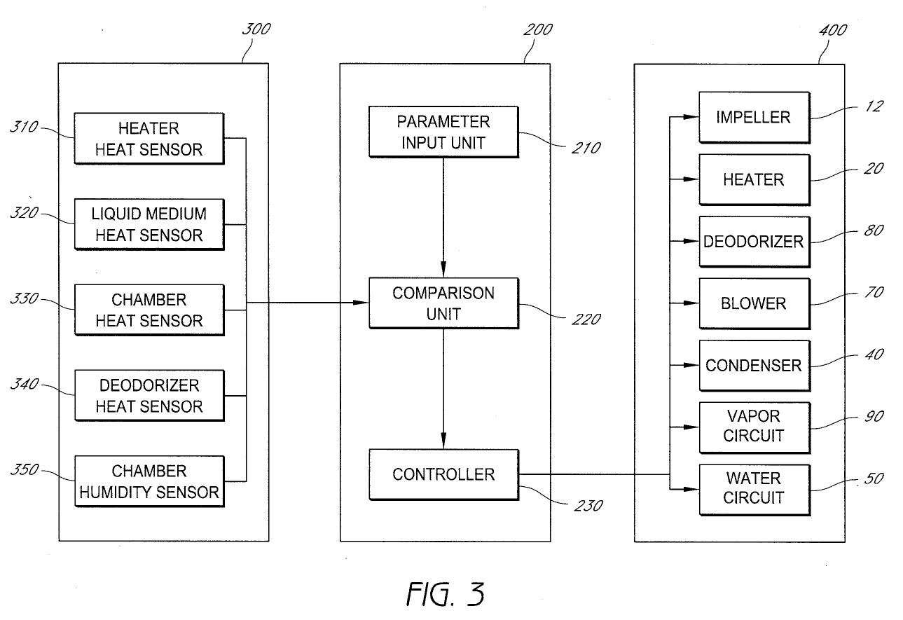

Referring to a block diagram as shown in FIG. 3, the organic waste decomposition system (OWDS) includes a control unit 200, a control input 300, and system components 400. The control input 300 can send information relating to operating conditions of the OWDS to the control unit 200. The control unit 200 can analyze the information and send signals to control the system components 400 of the OWDS to operate under desired conditions.

In the illustrated embodiment, the control input 300 includes a plurality of sensors 310, 320, 330, 340, and 350 that monitor the process of the OWDS and send signals to the control unit 200. The control unit 200 can analyze the signals from the control input 300 and control the operation of the OWDS by controlling at least some of the components of the system components 400.

In the illustrated embodiment, the control unit 200 includes a parameters input unit 210, a comparison unit 220, a controller 230, and a timer 240. In one embodiment, the parameter input unit 210 includes a control panel or an interface in which a user of the OWDS inputs data or various parameters or reference values for operating for the OWDS, such as desired temperature in the decomposition chamber 10, desired humidity in the decomposition chamber 10, desired flow rate of the blower 70, etc. The parameter input unit 210 can also set the time for decomposing the organic waste within the OWDS, such as from about 18 hours to about 24 hours. The information from the parameter input unit 210 is provided to the comparison unit 220. The parameter input unit 210 can include a computer, a solid state relay circuit, etc.

The comparison unit 220 receives input values from the parameter input unit 210 and compares them to the signals from the control input 300. The signals from the control input 300 can contain data relating to operating conditions or status of the OWDS during processing. The comparison unit 220 can analyze the signals from the control input 300 with the reference values from the parameter input unit 210 to determine if the signals from the control input 300 are within range of the reference values of the parameter input unit 210. If the signals are not within the pre-determined range, the comparison unit 220 sends control commands or signals to the controller 230 to control at least some of the system components 400 of the OWDS to meet the desired operating conditions. In turn, the controller 230 send control signals to appropriate system component 400 to adjust operating condition accordingly.

In one embodiment, the control input 300 includes a heater heat sensor 310, a liquid medium heat sensor 320, a decomposition chamber heat sensor 330, a deodorizer heat sensor 340, and a decomposition chamber humidity sensor 350. The heater heat sensor 310 can monitor the temperature of the heating member 21 of the heater 20 (of FIG. 1.) The liquid medium heat sensor 320 can monitor the temperature of the liquid medium 22 of the heater 20. The decomposition chamber heat sensor 330 can monitor temperature inside the decomposition chamber 10. The deodorizer heat sensor 340 can monitor temperature inside the deodorizer 80. The decomposition chamber humidity sensor 350 can monitor the relative humidity inside the decomposition chamber 10. The control input 300 is electronically connected to the comparison unit 220 and provides the sensed information as signals to the comparison unit 220.

In one embodiment, the system components 400 include the impeller 12 within the decomposition chamber, the heater 20, the deodorizer 80, the blower 70, the condenser 40, the vapor circuit 90, and the water circuit 50. The system components 400 are electronically connected to the controller 230 to be controlled by the controller 230. The impeller 12 can be controlled to determine the speed and/or direction of the rotation of the impeller 12 of the decomposition chamber 10. The heater 20 can be controlled to determine the amount of heat generated by the heater 20 and, consequently, transferred to the decomposition chamber 10. The deodorizer 80 can be controlled to determine proper temperature inside the deodorizer 80 to active the catalysts 82 in order to remove odor or smell that can be given off by the decomposing organic waste. The blower 70 can be controlled to determine the flow rate of the steam, and/or vapor from the decomposition chamber 10 through the condenser 40. The condenser 40 can be controlled to determine how much the cooling fans 42 of the condenser 40 can blow external air to the cooling pipes of the condenser 40 in order to precipitate water from the steam flowing therethrough. The vapor circuit 90 can be controlled to determine how much external air along with the vapor from the deodorizer 80 can be provided to the decomposition chamber 10 and/or the liquid medium 22. The water circuit 50 can be controlled to determine how much water from the water tank 55 and/or external water source can be provided back to the decomposition chamber 10, to the filter 30, or discarded out.

### Flow of Water and Air

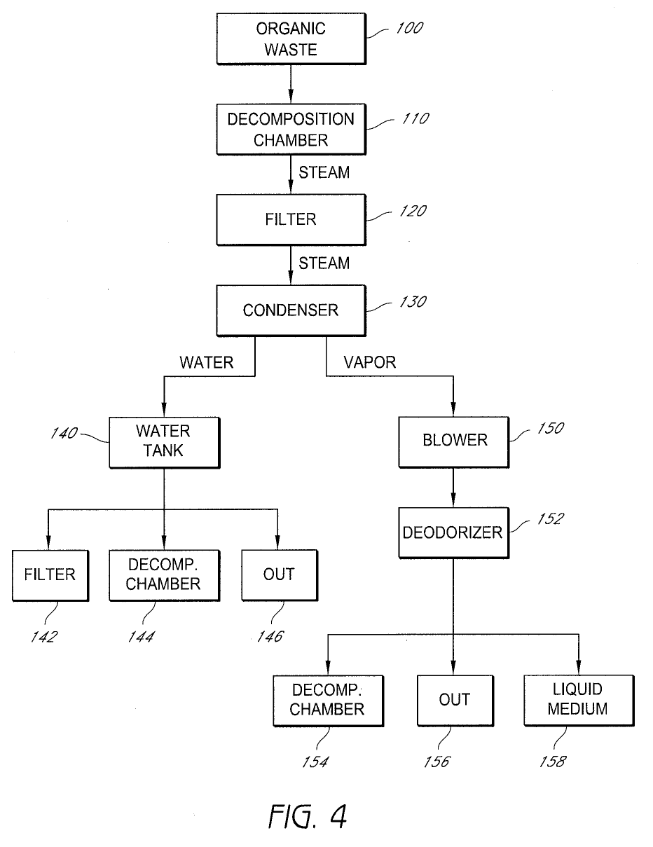

FIG. 4 illustrates flow of water and air during an organic waste decomposing process by the OWDS. In the illustrated embodiment, each block represents a stage during flow of water (moisture) and air in different components of the OWDS. Initially in block 100, moisture is kept in organic waste before the process. As the process begins the moisture comes out of the organic waste into the decomposition chamber 20. The moisture and steam in the decomposition chamber 10 then is flown through filter 30, to condenser 40 where at least some of it precipitates into water. The water from the condenser 40 moves to water tank 55. Then the water in the water tank 55 can be separated to the filter 30 to flush the filter 30, the decomposition chamber 10, and discarded outside. Some vapor passes from the condenser 40 to the blower 70, move to the deodorizer 80, and separate and move to decomposition chamber 10 and discarded outside.

In one embodiment, block 100 represents initial moisture content of the organic waste loaded into the decomposition chamber 10 (of FIG. 1.) In one embodiment, the decomposition chamber 10 uses heat and moisture released from the organic waste itself to decompose the organic waste. The decomposition chamber 10 is configured to decompose organic waste therein by heating the organic waste to release moisture and continue heating the organic waste to remove moisture and decompose without burning the organic waste. The heat needed to heat the organic waste can be provided from the heater 20 that can be at least partially surrounding the decomposition chamber 10. As the temperature inside the reaction chamber 10 (of FIG. 1) rises, the moisture within the organic waste starts to evaporate. The evaporated moisture or steam starts to build inside the reaction chamber 10.

Block 110 represents the steam in the reaction chamber 10. The OWDS maintains the air within the decomposition chamber 10 humid with the steam for at least part of the process of decomposing organic waste. The steam in the decomposition chamber 10 can be blown through the filter 30 (of FIG. 1) by the blower 70 (of FIG. 1). Block 120 represents the steam passing through the filter 30. The steam can be blown towards the condenser 40 (of FIG. 1) by the blower 70. At least some of the debris in the steam can be filtered by the filter 30. Block 130 represents the steam in the condenser 40. In the condenser 40, at least part of the steam can be separated into water and air. The water from the steam can be collected in the water tank 50 (of FIG. 1) and the less humid air after the condenser 40 can be blown through the blower 70.

Block 140 represents the water in the water tank 55. In some embodiments, at least some of the water in the water tank 55 can be pumped by the water pump 60 (of FIG. 1) to reuse or recycle the water within different units of the OWDS. In some embodiments, water can be used to flush the filter 30 and wash off at least some of the debris on the screen of the filter 30. Water flushed through the filter 30 can be represented by block 142. In some embodiments, some of the water in the water tank 55 can be returned to the organic waste in the reaction chamber 10 in order to provide enough moisture in the organic waste to prevent them from burning. The water pumped back to the reaction chamber 10 can be represented by block 144. In some embodiments, some water in the water tank 55 can be discarded from the OWDS. Block 146 represents the water drawn outside of the OWDS.

Block 150 represents the vapor blown through the blower 70 from the condenser 40. The vapor through the blower 70 can be provided to the deodorizer 80 (of FIG. 1). Block 152 represents the vapor from the blower 70 through the deodorizer 80. In one embodiment, the flow of the vapor can be controlled by the vapor circuit 90 (of FIG. 1). Block 154 represents that at least some of the vapor from the deodorizer 80 can be blown back to the decomposition chamber 10 (of FIG. 1) to recycle the heat retained within the air from the deodorizer 80. Block 156 represents some of the air from the deodorizer 80 can be blown outside the OWDS. Block 158 represents some of the air is returned to heat the liquid medium 22.

### SUMMARY

The system decomposes organic waste in a decomposition chamber without use of enzymes, additives, or microorganisms. In one embodiment, the system decomposes organic waste within 24 hours and deodorizes the odor of decomposing organic waste during decomposition process. The system provides sufficient heat and operating conditions to evaporate moisture from the organic waste without burning the organic waste. The byproduct of the organic waste after decomposition process by the system is substantially homogeneous material that is reduced in volume compared to the organic waste. The system reuses or recycles water and heat used in the system for different processes in the system. The system includes a blower that provides flow of the moisture inside the system.

What is claimed is:

1. An organic waste decomposition system comprising:
a decomposition chamber configured to decompose organic waste therein such that the decomposition chamber heats the organic waste to release moisture therefrom and continue to heat the moisture and organic waste to decompose, whereby steam is generated in the decomposition chamber;
a condenser in fluid communication with the decomposition chamber via a conduit and configured to precipitate water from the steam passing therethrough; and
a blower in fluid communication with the decomposition chamber and the condenser, the blower being configured to flow steam from the decomposition chamber to the condenser, wherein the blower is set to flow the steam at a flow rate such that a relative humidity within the decomposition chamber is maintained with less than about 10% variation for at least several hours.

2. The system of claim 1, wherein the decomposition chamber has an interior volume, wherein the blower is set to create the flow rate, which is from about 2 to about 4 times the interior volume for a minute.

3. The system of claim 1, further comprising an air inlet configured to receive and send ambient air to the decomposition chamber.

4. The system of claim 3, wherein the blower and the air inlet is set to create flow rate of steam and send ambient air to the decomposition chamber, respectively, to maintain the relative humidity inside the decomposition chamber at about 90% for at least several hours.

5. The system of claim 1, wherein the blower is configured to flow and discharge at least part of the steam from the condenser.

6. The system of claim 1, wherein the blower is configured to flow at least part of the steam from the condenser to the decomposition chamber.

7. A method of operating an organic waste decomposition system of claim 1, the method comprising:
decomposing organic waste in a decomposition chamber such that the decomposition chamber heats the organic waste to release moisture therefrom and continue to heat the moisture and organic waste to decompose, whereby steam is generated in the decomposition chamber;
condensing the steam passing through a condenser in fluid communication with the decomposition chamber via a conduit to precipitate water; and
flowing the steam from the decomposition chamber to the condenser with a blower in fluid communication with the decomposition chamber and the condenser, wherein the blower is set to flow the steam at a flow rate such that a relative humidity within the decomposition chamber is maintained with less than about 10% variation for at least several hours.

8. The method of claim 7, wherein the decomposition chamber has an interior volume, wherein the blower is set to create the flow rate, which is from about 2 to about 4 times the interior volume for a minute.

9. The method of claim 7, further comprising receiving and sending ambient air to the decomposition chamber with an air inlet.

10. The method of claim 9, wherein the blower and the air inlet is set to create flow rate of steam and send ambient air to the decomposition chamber, respectively, to maintain the relative humidity inside the decomposition chamber at about 90% for at least several hours.

11. The method of claim 7, wherein the blower is configured to flow and discharge at least part of the steam from the condenser.

12. The method of claim 7, wherein the blower is configured to flow at least part of the steam from the condenser to the decomposition chamber.
